(19) United States
(12) Patent Application Publication
(10) Pub. No.: US 2010/0323918 A1
(43) Pub. Date: Dec. 23, 2010

(54) POLYMER SURFACE FUNCTIONALIZATION AND RELATED APPLICATIONS

(75) Inventors: Joseph Huang, Plainsboro, NJ (US); Yufeng Ma, Boston, MA (US); Malcom R. Kahn, Franklin Lakes, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(73) Assignee: Microdysis, Inc, Bordentown, NJ (US)

(21) Appl. No.: 12/866,861

(22) PCT Filed: Feb. 10, 2009
(86) PCT No.: PCT/US09/33621

§ 371 Date: Aug. 9, 2010

Related U.S. Application Data
(60) Provisional application No. 61/027,461, filed on Feb. 10, 2008.

(51) Int. Cl. C40B 40/00; C08G 77/38; G01N 33/00

(52) U.S. Cl. 506/13; 525/474; 422/68.1

(57) ABSTRACT

A method to assemble functional materials, such as nanomaterials, onto a polymer surface to create a corresponding functionalized surface involves creating a solution of the functional material, providing a sacrificial substrate, disposing the functional solution onto a surface of the substrate and then covering the substrate with a liquid polymer. The sacrificial substrate is then dissolved, leaving behind a functional surface embedded within the cured polymer. One specific aspect of the invention relates to the embedding of functionalized carbon nanotubes onto a polymer surface for creating a nano-engineered surface. Devices employing functional surfaces are disclosed that are suitable for the immobilization of enzymes, DNA, peptides, proteins, cells, catalyst, and/or other chemicals or molecules for chemical, biochemical, or biological analysis, reactions, filtration.

### CROSS-REFERENCE TO RELATED APPLICATIONS

This application claims the benefit of U.S. Provisional Patent Application No. 61/027,461, filed on Feb. 10, 2008.

### FIELD OF THE INVENTION

This invention relates to the creation of surface wetting properties and three-dimensional structures on a polymer surface and to achieve uniform, reproducible, stable, and sterically accessible surface functionalization. More specifically, this invention relates to modification of surface properties and the embedding of functional or functionalized chemical or biochemical groups onto polymer surfaces for the immobilization of enzymes, DNA, peptides, proteins, cells, catalysts, and/or other chemicals or molecules for chemical, biochemical, or biological analysis, reactions, and filtrations. One specific aspect of the invention relates to the embedding of nanomaterials onto a polymer surface for creating a nanoengineered surface.

### BACKGROUND OF THE INVENTION

Deliberately creating structures with nanofeatures is a scientific and technical challenge that has been considered for several centuries. More recently, the assembly of carbon nanotubes from as-grown randomly tangled states into well-ordered 3D surfaces has attracted considerable attention from researchers and engineers worldwide due to the particular properties of the carbon nanotubes and their importance for chemical, biomedical and engineering applications. For many applications, well-ordered and functionalized carbon nanotubes are highly desirable. However, providing such arrays remains a significant challenge that is still at the prototyping level.

Biomolecular microarrays and microfluidic devices are emerging as powerful tools for genomics, proteomics, and clinical assays, since they realize a parallel and high throughput analysis of important biological events. Controlling and modifying the surface properties of microstructures can be a powerful tool in the design, fabrication, and use of microsystems. Due to surface heterogeneity, for example of protein analysis, proteins readily adsorb on polymeric surfaces via various interactions, which adversely affects the performance of microarray and microfluidic devices made from plastics or other polymers. Thus, modification of surfaces from hydrophobic to hydrophilic and vice versa, or from protein adsorbing to non-fouling remains a great challenge. Surface functionalization and immobilization of molecules or biomolecules are major issues for successful microarray and microfluidic assays.

### SUMMARY OF THE INVENTION

Various aspects of the invention provide a straightforward and effective technique to provide functional surfaces, such as by embedding well-ordered nanomaterials, and in particular carbon nanotubes, onto a polymer surface, such as polydimethylsiloxane (PDMS), elastomer or silicone rubber, and plastics. Various embodiments will open the door to the next generation of sensors and reaction chambers due to the superior properties of highly functionalized carbon nanotubes.

In one aspect a method is disclosed for making a functional or functionalized surface. Nanomaterials are dissolved into a solvent to create a nanomaterial solution. Alternatively, a polymer solution with functional molecular groups or a molecular imprinting solution that may be made by polymerizing monomers with template molecules may be used as the functional solution to create functional surface. The nanomaterial solution is deposited onto a surface of a sacrificial material. The sacrificial material is placed into a cavity, such as a mold, and the cavity is filled with a liquid polymer. The polymer is allowed to cure, and the cured polymer is then removed from the cavity. The sacrificial material is dissolved from the cured polymer yielding a nano-engineered surface or functional surface on the polymer. The nano-engineered surface or functional surface may be further adapted to immobilize DNA, RNA, peptides, proteins, cells or other organic molecules or chemicals.

In various embodiments the solvent is an organic solvent, such as ethanol, methanol, acetonitrile (ACN), trifluoroethanol (TFE), isopropyl alcohol and combinations thereof. In various embodiments the functional material in the solvent comprises chemically processed nanomaterials, palmitic acid, polyhydroxystyrene, polyacrylic acid, polycarbonate resin, or combinations thereof. In other embodiments, the functional material comprises a molecular imprinting polymer solution comprising template molecules polymerized with monomers.

In various embodiments the nanomaterials are selected from single-walled carbon nanotubes, multi walled carbon nanotubes, nanowires, or nanoparticles. Preferred embodiments employ carbon nanotubes.

In various embodiments the sacrificial material is made from a water-soluble or a solvent-soluble material, and in particular from polyvinyl alcohol (PVA), starch, gelatin, synthetic polymers, colloid gels, or lipid materials. Dissolving the sacrificial material from the cured polymer may include dissolving the sacrificial material with water, acid, base, or other solvents.

In specific embodiments the polymer is selected from polydimethylsiloxane (PDMS), polyurethane, polydimethylsiloxane, polycarbonate, polypyrrole, resin, Teflon resin, epoxy, polymeric rubber, and polymeric plastic.

Devices comprising nano-engineered surfaces formed by the above method are also disclosed. Such devices include, for example, cuvettes, microtiter plates, and reaction or filtration vessels. Such devices will typically include an enclosure body, an inlet in the enclosure body, an outlet in the enclosure body, a fluidic channel in the enclosure body fluidly connecting the inlet to the outlet, and a nano-engineered surface in the fluidic channel. Embodiment devices may further include an orifice to control a flow rate of fluid within the fluidic channel. In embodiments such as reaction or filtration vessels, the fluidic channel may include a plurality of columns to increase both turbulence in the fluid flow and nano-engineered reaction surface areas.

In some embodiments a filling is disposed within the enclosure body, and it is the filling that defines the fluidic channel. In certain specific embodiments the filling is made from one or more of polydimethylsiloxane (PDMS), polyurethane, polydimethylsiloxane, polycarbonate, polypyrrole, resin, Teflon resin, epoxy, polymeric rubber, and polymeric plastic.

In devices that are employed for optical detection purposes, such as cuvettes, the nano-engineered surface may be disposed on a sidewall of the fluidic channel that is substantially perpendicular to a detection light pathway.

In a specific embodiment cuvette, a fluid collection portion that removably connects to the enclosure body is provided. The fluid collection portion is adapted for fluidic communications with the outlet and comprises a reservoir for accepting fluid from the outlet. The fluid collection portion may be removed and the collected fluid re-introduced back into the inlet for further processing.

An embodiment microtiter device includes a body, and a plurality of reservoirs disposed in the body. Each of the plurality of reservoirs includes a nano-engineered surface.

These and other aspects will be described in more detail in the following.

### BRIEF DESCRIPTION OF THE DRAWINGS

FIGS. 1A and 1B are Atomic Force Microscopy (AFM) images of a blank sacrificial substrate, in which FIG. 1A shows a top view morphology and FIG. 1B provides a three-dimensional surface profile.

FIGS. 2A and 2B are Atomic Force Microscopy images of carbon nanotubes on a sacrificial substrate, in which FIG. 2A shows a top view morphology and FIG. 2B provides a three-dimensional surface profile.

FIGS. 3A and 3B are Atomic Force Microscopy images of three-dimensional surface profiles, in which FIG. 3A shows a blank PDMS surface and FIG. 3B shows embedded functionalized carbon nanotubes on a PDMS surface.

FIG. 4 is a perspective view of an embodiment cuvette-shaped device with a filling region and a channel inside the filling region.

FIG. 5 is a photograph of the embodiment cuvette show in FIG. 4.

FIG. 6 is a side view of the embodiment cuvette shown in FIG. 4.

FIG. 7 is another side view of the embodiment cuvette shown in FIG. 4.

FIG. 8 is a side view of another embodiment cuvette.

FIG. 9 is another side view of the cuvette of FIG. 8.

FIG. 10 is a side view of yet another embodiment cuvette.

FIG. 11 is a perspective view of a further embodiment cuvette.

FIG. 12 is a side view of the embodiment cuvette shown in FIG. 11.

FIG. 13 is a top view of an embodiment microtiter plate.

FIG. 14 is a sectional view of a microtiter well depicted in FIG. 13.

FIG. 15 shows a sectional view of another embodiment of a microtiter well.

FIGS. 16A and 16B show fluorescence images for arsenic detection. FIG. 16A is an image of fluorescence intensities in two wells after coupling of a fluorescence tag, and FIG. 16B shows fluorescence intensities after an arsenic solution has been applied to the wells for five minutes.

FIG. 17 is a perspective view of an embodiment fluidic channel with columns inside the channel and nanomaterials embedded on both the channel and the column surfaces.

FIG. 18 is side view of the embodiment shown in FIG. 17.

### DETAILED DESCRIPTION

Definitions

The term “nanomaterial” as used in the following refers to a material with morphological features smaller than a one tenth of a micrometre in at least one dimension. Such materials include single-walled or multi-walled carbon nanotubes, nanowires, or nanoparticles.

The term “functional solution” as used in the following refers to an aquatic or organic solution in which a functional material, which may be a chemical compound, a polymer, or a nanomaterial, is dissolved or dispersed. Such functional materials carry, or may be modified or activated to carry, one or more functional groups, such as, carboxyl groups (COOH), amino groups (NH2), or hydroxyl groups (OH). Functional materials may also include molecularly imprinted materials that are used to create imprints (recognition elements) on a polymer to create a corresponding functionalized surface. Hence, for example, a “carbon nanotube solution” refers to an aquatic or organic solution with carbon nanotubes dispersed therein. Non-limiting examples of functional materials include single-walled carbon nanotubes, multi-walled carbon nanotubes, nanowires, or nanoparticles, or a mixture of nanomaterials with other materials with functional chemical or biochemical groups, such as palmitic acid, polyhydroxystyrene, polyacrylic acid, and polycarbonate resin.

The term “sacrificial mold material” refers to a mold material made from one or more soluble materials. A “soluble material” refers to a synthetic polymer, colloid gel, starch, or lipid material that is solid, preferably at room temperature, but is soluble upon contact with a suitable solvent. Examples of soluble materials include polyvinyl alcohol (PVA), starch, wax, and gelatin. A sacrificial mold material can be cast as a sheet or molded to a certain shape that is complementary to a structure that is to be fabricated inside a device. A sacrificial mold material can be removed from the device body after the device has been molded.

The term “functional surface” or “functional spot” refers to an area where the surface property is modified to have a particular or desired function, for example to have COOH functional groups, or to exhibit surface wetting properties, such as hydrophilicity/hydrophobicity, or binding properties, which are different to the surfaces in other regions.

The following provides a description of how to create an embodiment functional surface on a polymer surface. As a first step, a functional solution may be made or provided. In the following, by way of example with preferred embodiments, poly(4-vinylphenol) and carbon nanotubes are discussed. However, it will be appreciated that other types of chemical compounds, polymers, nanomaterials or functional solutions may be used.

### Example 1

Creation of a poly(4-vinylphenol)ethanol solution: poly(4-vinylphenol), also called polyvinylphenol or PVP, is a plastic structurally similar to polystyrene. It includes a hydroxyl group (OH) in its chemical structure. With such functional groups, the functional surface can be modified with OH groups and made hydrophilic. Poly(4-vinylphenol) is soluble in ethanol. To make a 1% poly(4-vinylphenol) ethanol solution 10 ml, 100 mg of poly(4-vinylphenol) is added to a 10 ml ethanol solution. Poly(4-vinylphenol) can be totally dissolved by sonicating for 5 minutes at room temperature.

### Example 2

A carbon nanotube solution: Generally, carbon nanotube solutions are commercially-available, and can be purchased as water soluble or solvent soluble solutions, and may be provided with or without functional groups. Carbon nanotube solutions in which the carbon nanotubes do not have functional groups may be considered “pristine” carbon nanotubes, whereas those with functional groups may be considered “chemically processed” carbon nanotubes. Carbon nanotubes may also be processed to be water soluble, and thus provide an aquatic nanotube solution, by way of a variety of macromolecules (single-stranded/double-stranded DNA, RNA, Chitosan, and glycopolymer, etc.). If the functional solution is preferred to be organic solvent-based, the water may be replaced by the desired solvent by way of, for example, centrifuge dialysis. Preferred solvents include a combination of one or more of ethanol, methanol, acetonitrile (ACN), trifluoroethanol (TFE), and isopropyl alcohol.

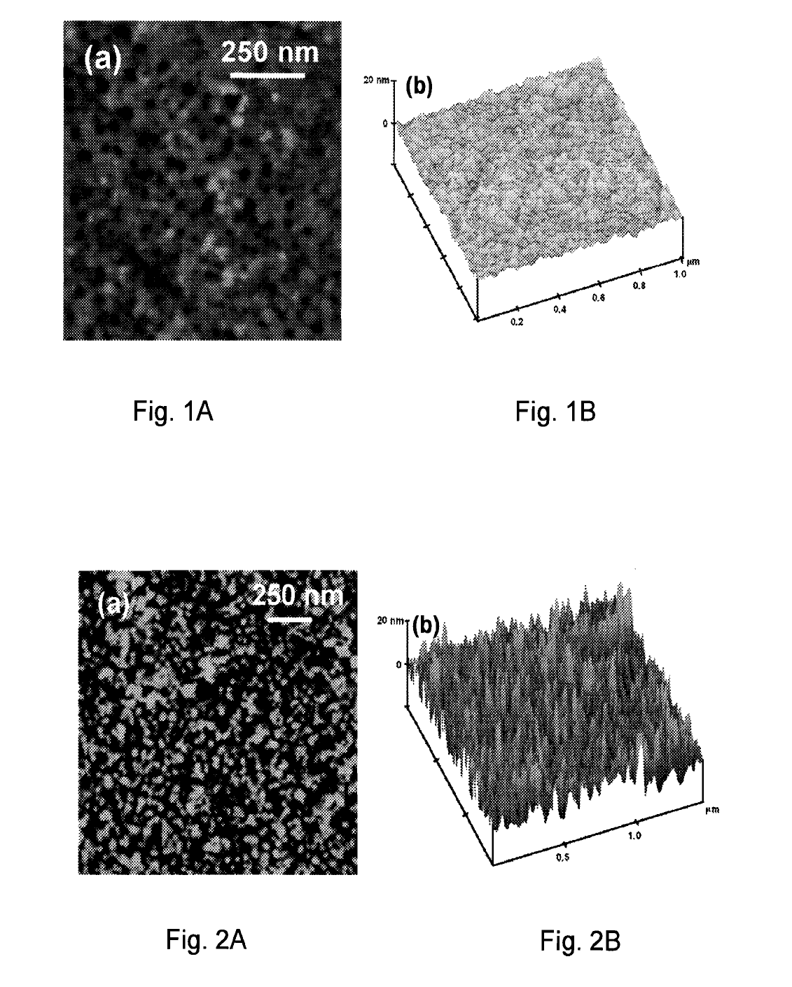

In various embodiments, a sacrificial substrate may then be created or provided for another step of an embodiment process. The sacrificial substrate may be made from a water soluble or solvent soluble material. The water soluble or solvent soluble material may be cast as a sheet with a porous surface, although any shape is possible, such as rod-shaped or even irregular shapes. The pore size is preferred to be 15 nm in diameter, but in other embodiments may range from 10 nm to 1000 nm in size. By way of example, when the sacrificial substrate is cast the pore size on the substrate can be controlled by the ratio of water to solvent, in which the sacrificial mold material is dissolved. As a preferred embodiment, a sacrificial substrate is fabricated from a sacrificial mold material. The sacrificial mold material can be a part of a mold, or may be suspended in the cavity of a mold or device. FIGS. 1A and 1B show Atomic Force Microscope (AFM) images of a blank sacrificial substrate. FIG. 1A depicts the top view morphology of the blank substrate, while FIG. 1B shows a three-dimensional surface profile of the blank substrate. AFM provides a very high-resolution scanning probe microscope with demonstrated resolutions of fractions of a nanometer. In FIG. 1, numerous holes can be observed on the blank surface of the sacrificial substrate, the diameters of which are about 15 nm on average. The roughness of the blank surface is less than 5 nm, which is indicated by FIG. 1.

The functional solution, such as the above-described carbon nanotube solution, is contacted with the sacrificial mold material at one or more locations corresponding to where a nano-engineered or functional surface is desired. For example, one or more droplets of the functional solution may be deposited onto the surface of the sacrificial mold material at positions corresponding to the desired locations of one or more functional surfaces in the finished product. One or more functional solutions may be used, each with its own functional properties. The one or more functional solution(s) may be disposed on any desired surface of the sacrificial mold, and may be disposed on more than one surface, or in more than one region. For example, one type of functional solution may be disposed on one side of the sacrificial mold and/or another type on another or both sides of the sacrificial mold. After the droplet has dried, a uniform functional spot is created, such as a carbon nanotube spot. FIGS. 2A and 2B are AFM images of carbon nanotubes on the sacrificial mold surface. FIG. 2A shows the top view morphology of the nanotube spot surface, and FIG. 2B shows a three-dimensional surface profile of the nanotube spot surface. As shown by FIG. 2A, most of carbon nanotubes appear slightly aggregated (˜10 nm in diameter), with some large bundled structures (˜40 nm in diameter). The carbon nanotubes may be found to be vertically and uniformly self-assembled on the sacrificial substrate, as indicated by FIG. 2B.

After the formation of uniform functional materials, such as the example here of carbon nanotubes, onto the sacrificial substrate, by way of example a simple open-top mold can be assembled with the sacrificial substrate and side walls to cast a device with a functional surface. The use of sacrificial molds in the formation of components or devices is described in greater detail in U.S. Pat. No. 7,125,510, but the outlines of the process may be briefly described as follows. A liquid polymer, preferably a polydimethylsiloxane (PDMS) mixture comprising a precursor and a curing agent, or other liquid polymers such as polyurethane, polycarbonate, polypyrrole, resin, Teflon resin, epoxy, polymeric rubber, or polymeric plastic, may be poured into the mold onto the sacrificial substrate and left to cure. The internal shape of the mold provides the desired shape of the component or device, while the position and shape of the sacrificial mold substrate may correspond to, for example, the shape of interior regions within the component or device. After disassembly of the side walls from the sacrificial substrate, the sacrificial substrate is then dissolved or removed from the cured polymer using water or a suitable solvent. The functional surface once present in the now-dissolved sacrificial mold substrate leaves a corresponding functional surface in the cured polymer.

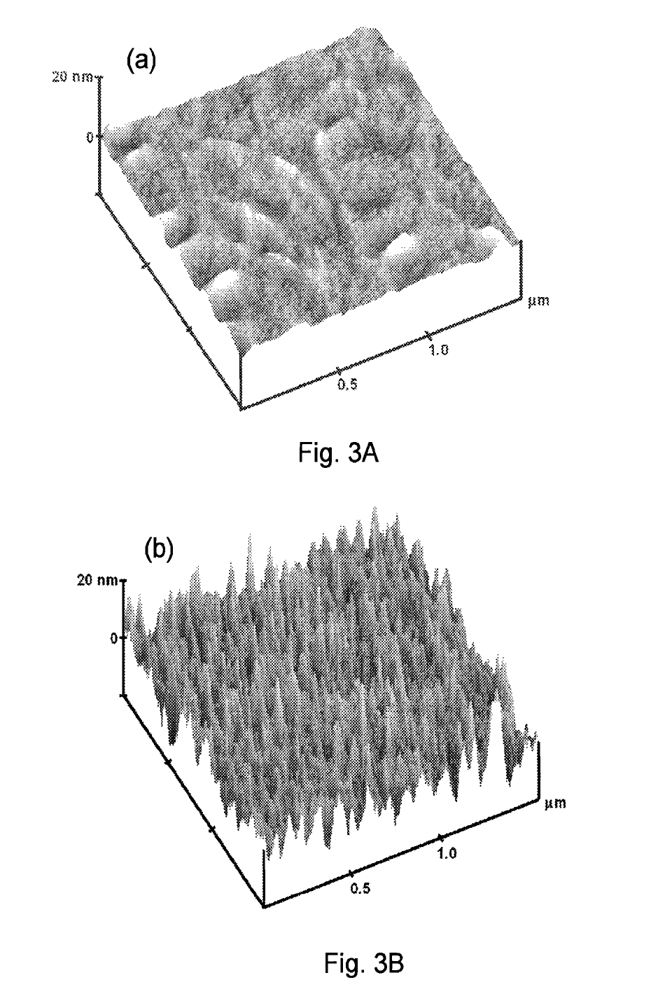

The morphology of the embedded functional material in a polymeric surface, such as a PDMS surface, can also be characterized by AFM or SEM (scanning electron microscope). FIG. 3B clearly shows that well-controlled nanotube architectures are transferred from the sacrificial substrate onto an embodiment PDMS matrix through this straightforward method. These nano-engineered structures precisely retain their original alignment, shape, and size inside the PDMS matrix. The average height of the embedded carbon nanotubes on PDMS is around 25 nm, as indicated by FIG. 3B. Hence, a nano-engineered surface is created that is complementary to the shape of the sacrificial substrate.

As shown by FIG. 3B, embodiment nano-engineered or functional surfaces may exhibit exceptional uniformity. By way of comparison, an AFM image of a blank PDMS surface is shown in FIG. 3A.

Without being bound by theory, it is believed that one possible reason for the vertical and uniform assembly of the nanomaterials, such as carbon nanotubes, on a polymeric surface, such as PDMS, is the capillary force as applied to, for example, the carbon nanotube solution. A morphological study of a blank sacrificial substrate, such as that shown in FIG. 1A, shows numerous holes of about 15 nm in diameter on the surface of the substrate. These small holes may generate capillary forces with respect to the nanomaterial solution deposited on the surface of the substrate. These capillary forces may then pull the nanomaterials, such as carbon nanotubes, suspended in the solution into the holes. Once the solution dries, the nanomaterials remain entrapped in the holes and assembled vertically on the surface of the sacrificial substrate.

Embodiments may provide various advantages, some of which include:

1. Uniformly and vertically assembled nanomaterials, such as carbon nanotubes, that significantly increase the surface area of a region.

2. Before assembly, nanomaterials can be processed to have functional groups on their surfaces, such as placing carboxyl or amino groups on carbon nanotubes. As a result, nano-engineered surfaces formed by the nanomaterials may include the functional groups and hence may provide a versatile substrate for sensing, bonding, coupling, condensing, or filtering molecules or biomolecules.

3. Embodiment processes are simple and inexpensive to perform.

Considering the unique properties of nanomaterials, and carbon nanotubes in particular, the ultra-dense functional groups that may be provided on the nanomaterials, as well as flexibility and transferability issues, various embodiments of the invention may open new avenues for many applications.

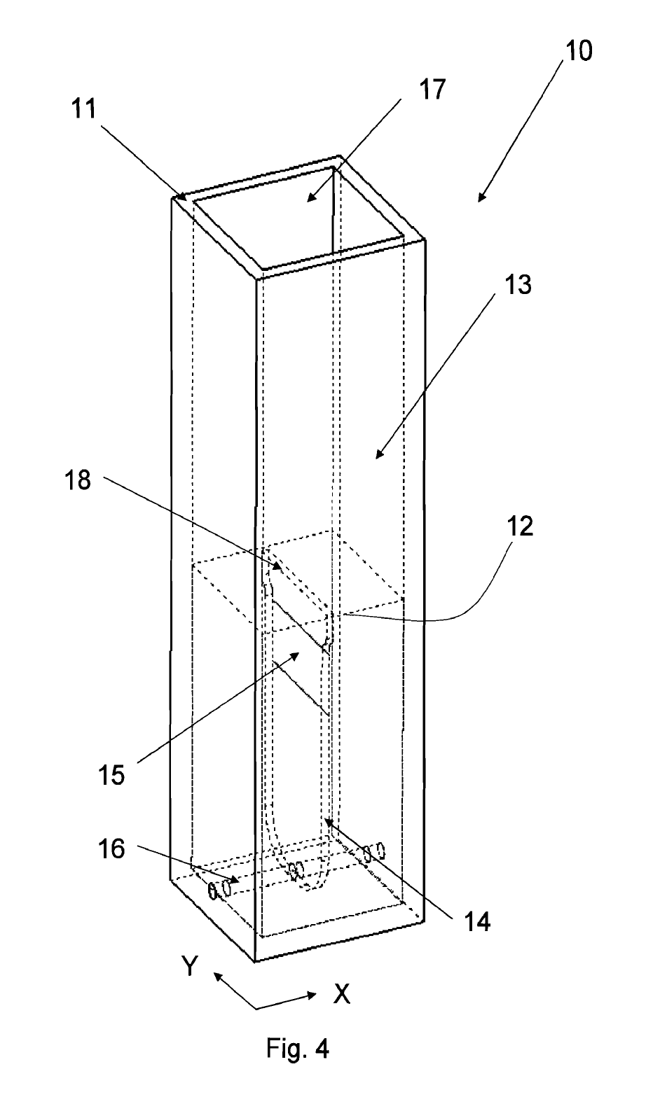

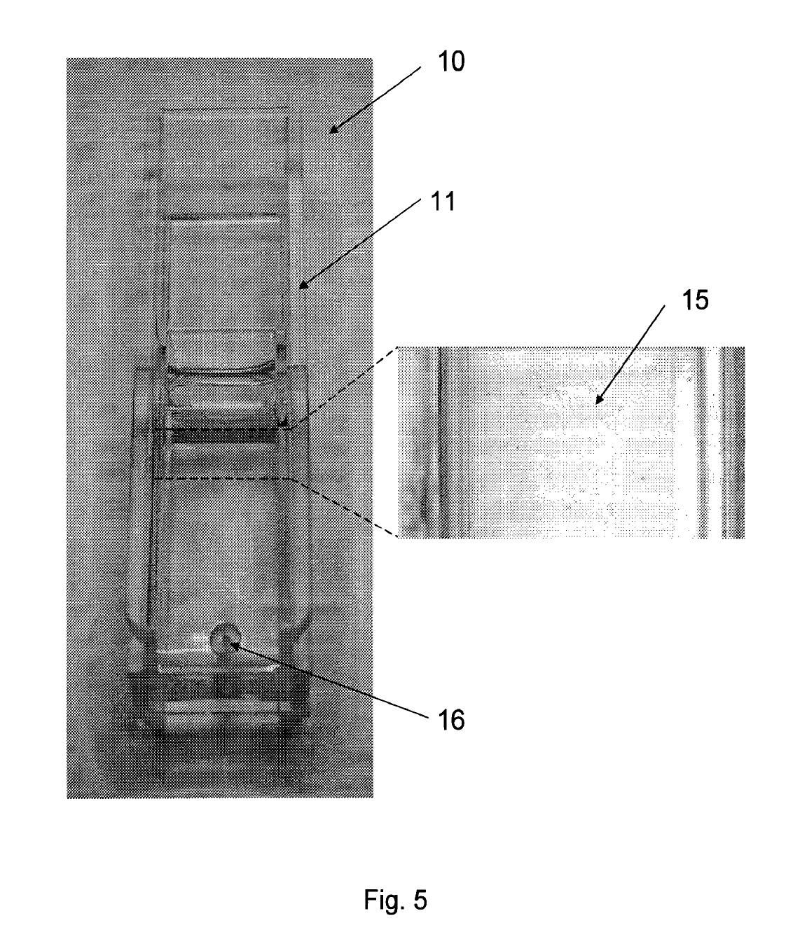

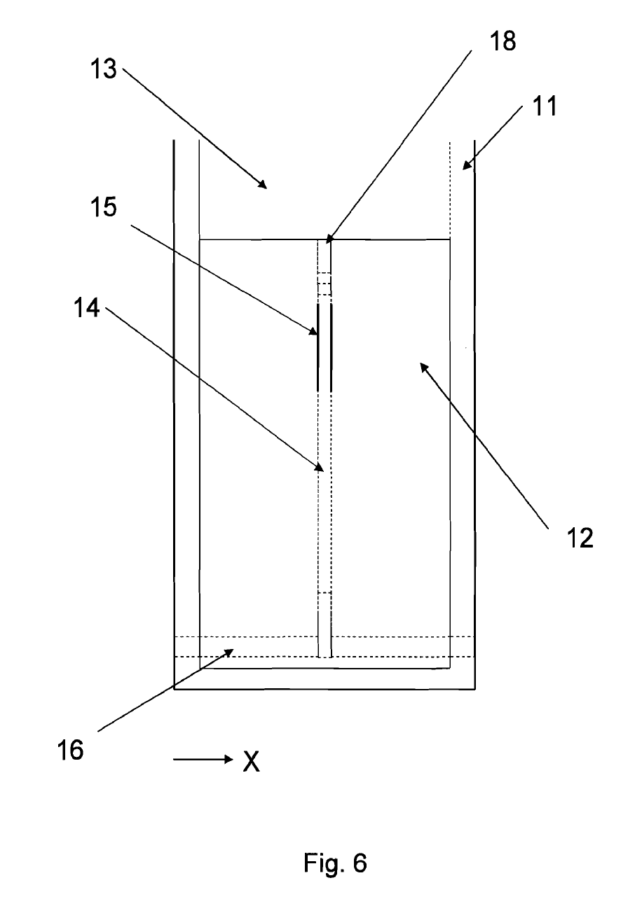

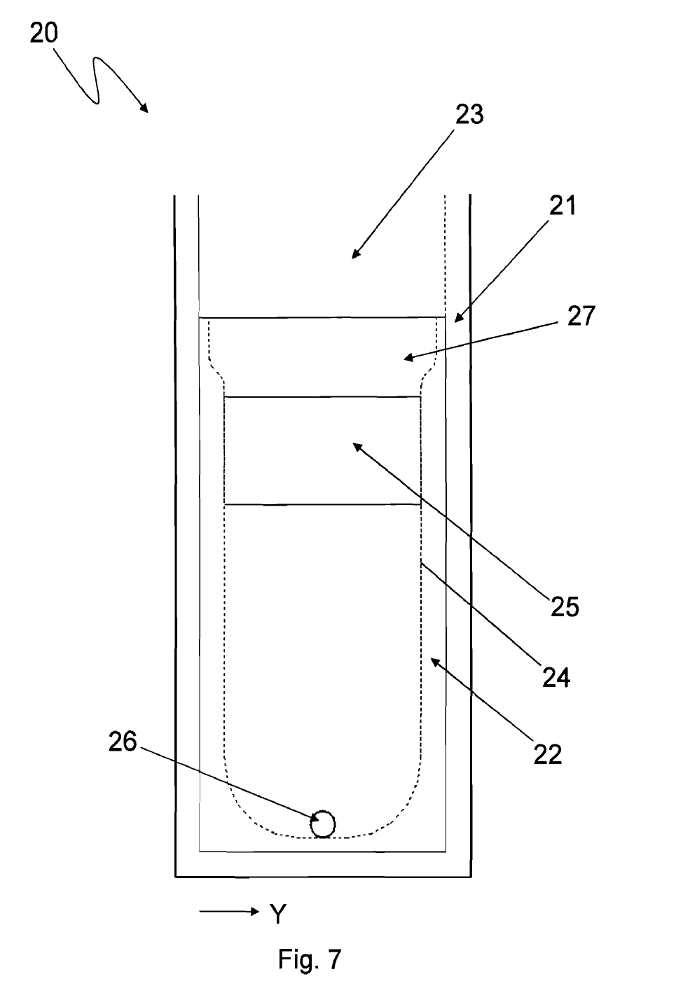

Certain embodiment nano-engineered or functionalized surfaces may be used for chemical and biochemical analyses and reactions, as well as sample injections. FIG. 4, for example, is a perspective view of an embodiment cuvette 10 suitable for use in a spectrometer of spectrophotometer. FIG. 5 is a photograph of the embodiment cuvette shown in FIG. 4, while FIG. 6 and FIG. 7 provide side views of the embodiment cuvette.

A cuvette is a type of laboratory vessel, usually a small tube of circular or square cross section, sealed at one end, made of plastic, glass, or optical grade quartz, and designed to hold samples for spectroscopic experiments. In FIG. 4, the cuvette 10 includes an enclosure body 11 with an opening 17 on the top of the enclosure 11. Inside the enclosure 11 a filling 12 is disposed, which may go to about half of the height of the cuvette 10. The filling 12 can be a material that is optically transparent, such as PDMS or other liquid polymers. The filling 12 is designed to include a slot fluidic channel 14 that fluidly connects an opening 18 on the top of the filling 12 with an outlet orifice 16 at the bottom of the filling 12. In a specific embodiment, the width of the channel 14 along the X direction shown in FIG. 4, which may be the optical beam pathway for detection purposes, is 0.1 mm; in various other embodiments the width may range from 0.01 to 5 mm. The aspect ratio of the slot 14 may range, for example, from 1:10 to 1:100. The width of the channel 14 along the Y direction indicated in FIG. 4 may be 10 mm, but in other embodiments may range from 1 to 10 mm. It will be appreciated that the shape of the fluidic channel can be square, circular or other shapes as desired.

By way of example, the cuvette 10 can be made by assembly of the enclosure body 11 with a strip of a sacrificial material and some alignment mold components that hold the sacrificial strip straight in the enclosure body 11. The thickness and the width of sacrificial material may be made equal to the dimensions of the channel 14 to be created in the cuvette 10. The functional solution, such as a carbon nanotube solution, can be dip-coated onto the sacrificial strip then transferred and embedded on the sidewall of the channel 14 after the sacrificial strip is removed from the filling 12, which is poured into the enclosure body 11 around the sacrificial mold and allowed to cure. Alternatively, for example, the functional solution may be pipetted onto the sacrificial strip at the desired location or locations of the corresponding functional spots. The functional surface or material, such as carbon nanotubes, may be embedded on one side of the walls, on both sides of the walls, or on a portion of the sidewalls, for example at the detection level (beam height) of a standard spectrometer, as indicated by arrow 15. Furthermore, two or more different functional surfaces, such as one with carboxyl groups, and another with amino groups, can be embedded on the two sides of the channel wall respectively. Hence, multiple functional surfaces, each with different, respective functionalized properties, may be provided in a single device, one of which may be used as internal control. The upper portion of the cuvette 10 forms a compartment 13 that can be used to hold a sample liquid. For detection purposes, the sample liquid is loaded into the compartment 13. Because of capillary forces and gravity, the sample liquid flows down from the opening 18 to the outlet orifice 16. The preferred diameter of the orifice 16 is 0.2 mm, but greater or smaller diameters are possible to control the flow rate of the sample liquid as it passes through the detection nanotube portion 15 in the channel 14. The nanotubes with functional groups on their surfaces and/or enzymes or molecules binding to the functional groups, sense the target ions or molecules in the sample. Then the sample-exposed cuvette 10 is read using any suitable equipment, which may include, for example, looking for absorbance or fluorescence by way of a spectrometer, a fluorescence microscope, or the like.

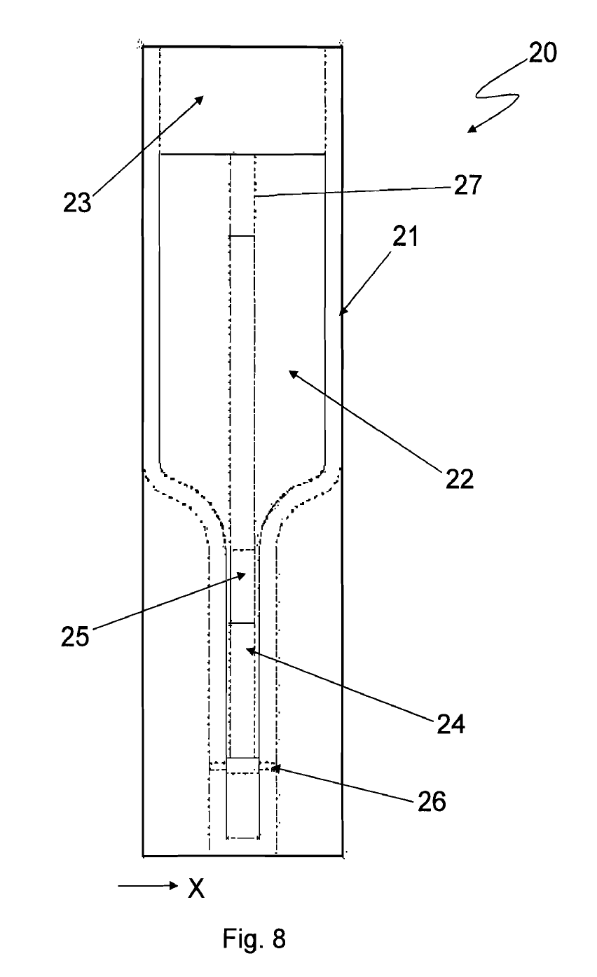

FIGS. 7 and 8 illustrate another embodiment cuvette 20. The cuvette 20 has a narrowed portion along the X direction, which may help with sample usages and increase detection sensitivities. The cuvette 20 includes an enclosure 21, a compartment 23 in the upper portion of the enclosure 21, and a filling 22 in the lower portion of the enclosure 21. A slot channel 24 and an outlet orifice 26 are disposed inside the filling 22 and fluidly connected to the compartment 23 via an opening 27. In preferred embodiments the opening 27 is funnel-shaped within the filling 22. The width of the channel 24 along the X direction (the optical path) may be 0.1 mm, but in other embodiments may range from 0.05 to 1 mm. The width of the channel 24 along the Y direction is 2 mm, but may range from 1 mm to the internal width of the enclosure 21. Carbon nanotubes or other functional materials can be embedded on one or more of the sidewall surfaces of the channel 24 by way of the procedures given above. The functional surfaces, such as are provided by carbon nanotubes, may be disposed on one of the sidewalls, on both sidewalls, or on one or more portions of the sidewalls, as shown by arrow 25 for a specific embodiment, which may be a region of the sidewall(s) at the detection beam height of a typical or target spectrophotometer.

For absorbance detection, the channel 24 is preferably perpendicular to the optical path. If the cuvette is to be used for fluorescence detection in a spectrophotometer, the channel 24 can be disposed within the filling 22 so that the channel 24 is oriented at an angle to the optical path, such as 45 degrees to the optical path.

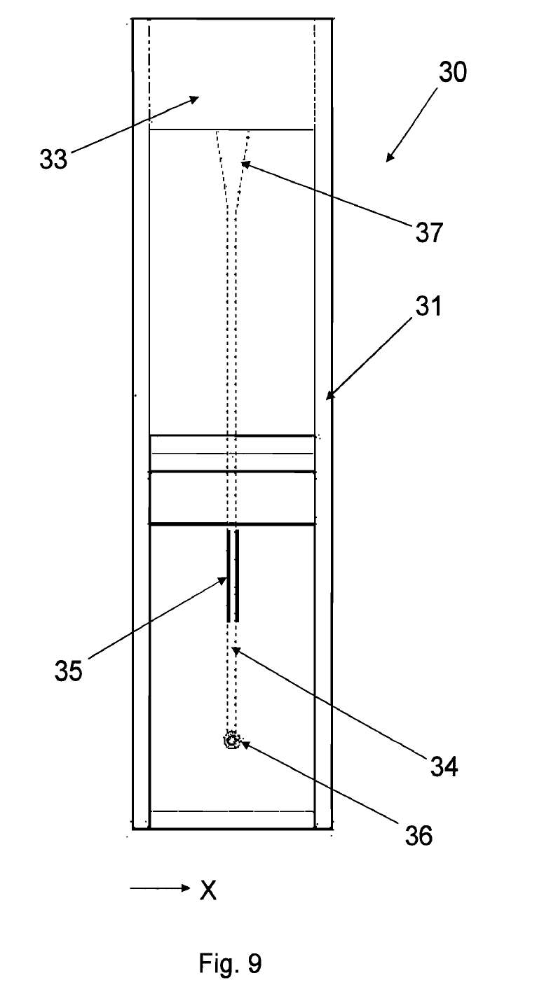

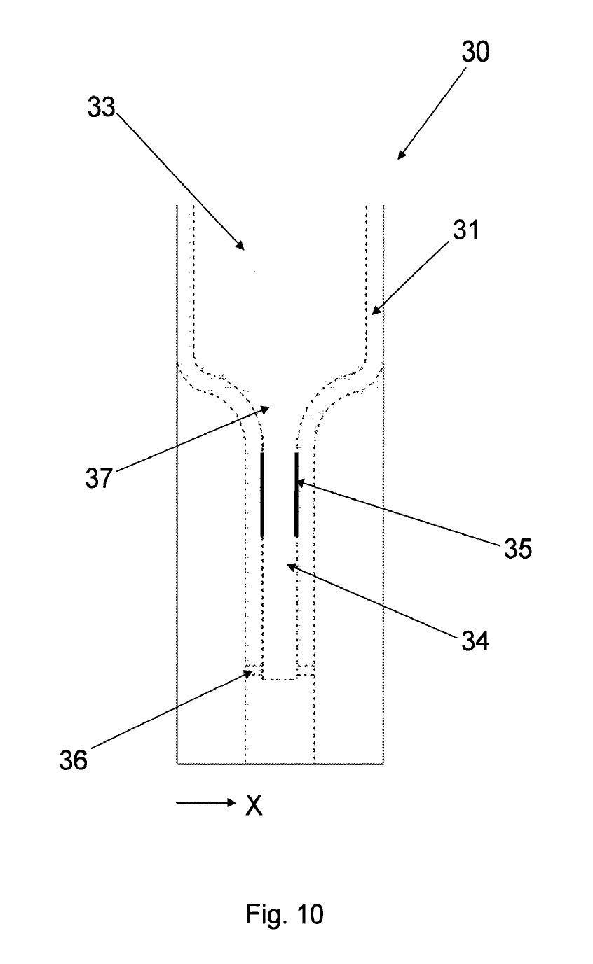

Another embodiment cuvette 30 is depicted in FIGS. 9 and 10, in which the nano-engineered or other functional surfaces are directly embedded at the detection position on the internal wall of the cuvette enclosure 31. The cuvette 30 includes the enclosure 31, a compartment 33 in the upper portion of enclosure 31, and a narrowed lower portion 34 of enclosure 31. An opening and an outlet orifice 36 are disposed within the lower portion 34. The opening 37 is funnel-shaped. The width of the narrowed lower portion 34 along the X direction (that is, the detection beam pathway direction) is 0.5 mm, but may range from 0.1 to 5 mm. Functional materials, such as carbon nanotubes, may be embedded on the internal wall of the narrowed portion 34 of the enclosure 31 by way of the procedures given above. The functional materials can be embedded on one side of the walls, on both sides of the walls or, as shown by arrow 35, in one or more regions at the detection beam height of a spectrophotometer.

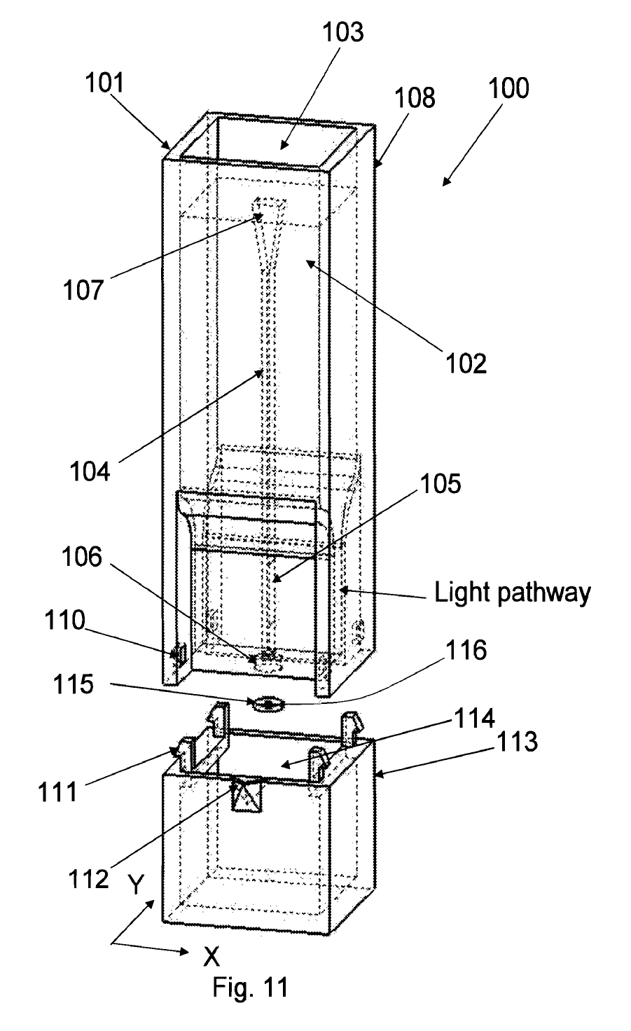

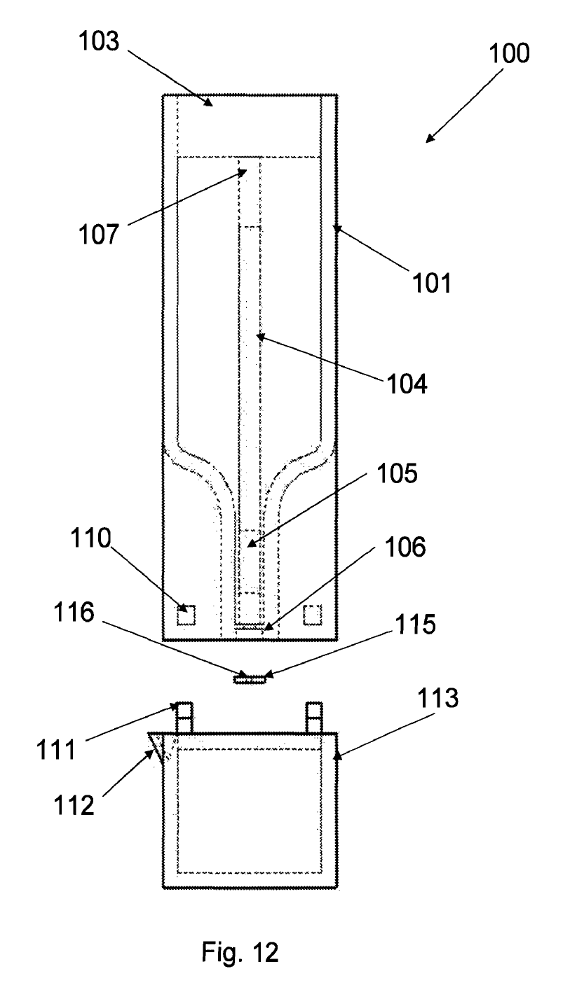

FIG. 11 is an exploded perspective view of yet another embodiment cuvette 100. FIG. 12 is an exploded side view of the cuvette 100. The cuvette 100 includes an upper detection portion 108, an orifice 115, and a lower liquid collection portion 113. The upper detection portion 108 may be a part of, or include components from, for example, cuvette embodiments 10, 20, or 30. The upper detection portion 108 includes an enclosure 101, a compartment 103 in the upper portion of the enclosure 101, and, for example, a filling 102. Inside the filling 102, or provided by the enclosure 101 itself, there is a channel 104 with an opening 107 on the top and an outlet orifice 106 at the bottom. For this embodiment, the width of the channel 104 along the X direction (that is, the detection beam pathway) is 0.1 mm, but may range, for example, from 0.01 to 5 mm. The width of the channel 104 along the Y direction is 2 mm, but may range from 1 to 10 mm for the narrowed portion of the cuvette 100. Functional materials, such as carbon nanotubes, are embedded on one or more of the sidewalls of the channel 104; for example, as shown by arrow 105, a region of embedded carbon nanotubes may be provided at the detection beam height of a spectrophotometer. In the upper detection portion 108 of the cuvette 100 there is a compartment 103 that can be used to hold a sample liquid. For measurement purposes, the sample liquid is loaded into the compartment 103. If the sample volume is small, the sample can be directly applied to the funnel opening 107 of the channel 104. Due to capillary forces and gravity, the sample liquid flows down from the opening 107 to the outlet 106 via the fluidic channel 104. The functional surfaces, such as nanotubes with functional groups on their surfaces and/or enzymes or molecules bonding to the functional groups, sense the targeted ions or molecules in the sample liquid as the fluid passes through the fluidic channel 104.

The orifice 115 with its internal diameter 116 controls the flow rate of the sample liquid as it passes the nanotube portion 105 in the channel 104. The sample liquid is collected in the reservoir 114 of the liquid collection portion 113. To increase detection sensitivity and usage of the sample, the collected sample can be poured back into the compartment 103 of the detection portion 108 for double or triple binding to the nanotube portion 105. A funnel 112 may be provided to assist in the pouring of the collected sample liquid. For easy attaching and detaching, the sample collection portion 113 or the detection portion 108 may include one or more click latches 111, such as on the four corners of the respective portion 113, 108. A corresponding hole or holes may be provided on the other portion 108, 113 to accept the latches 111.

The cuvette 100 may be read by a spectrometer for absorbance or fluorescence of the sample attached to the nano-engineered surface.

By way of a specific example, a nano-engineered cuvette for Fe3+ detection in a water solution is presented in the following.

Protocol for Preparation:

1. Rinse the channel in an embodiment cuvette with deionized (DI) water three times; the cuvette has, for example, a functional spot provided by chemically processed nanotubes with COOH functional groups.

2. Prepare 1 mL of 2 mM 1-ethyl-3-(3-dimethylaminopropyl)carbodiimide hydrochloride (EDC) and 5 mM N-hydroxysuccinimide (NHS) in 0.1M MES (2-(N-morpholino)ethanesulfonic acid) buffer solution. Add this mixture into the upper compartment of the cuvette and incubate for 30 min. During the incubation, keep refilling the upper compartment of the cuvette with the solution flowing from the outlet channel.

3. After incubation, rinse the cuvette with DI water three times.

4. Prepare 1 mL of 5 mM deferoxamine (DFO) water solution. Add the solution into the upper compartment of the cuvette and incubate for 1 hr. During the incubation, keep refilling the upper compartment of the cuvette with the solution flowing from the outlet channel.

5. After incubation, rinse the cuvette with DI water three times.

6. Dry the cuvette with N₂ gas and keep it for later use. The cuvette now has functional sensing molecules (DFO) on the carbon nanotubes for Fe3+ detection and can be used for subsequent testing procedures.

Protocol for Sensing Fe3+:

1. Take a baseline of the above dried cuvette using UV/VIS spectroscopy.

2. Add 1 mL of a sample solution with Fe3+ into the upper compartment of the cuvette and let it flow from the outlet channel.

3. Dry the exposed cuvette with N₂ gas and test using UV/VIS spectroscopy.

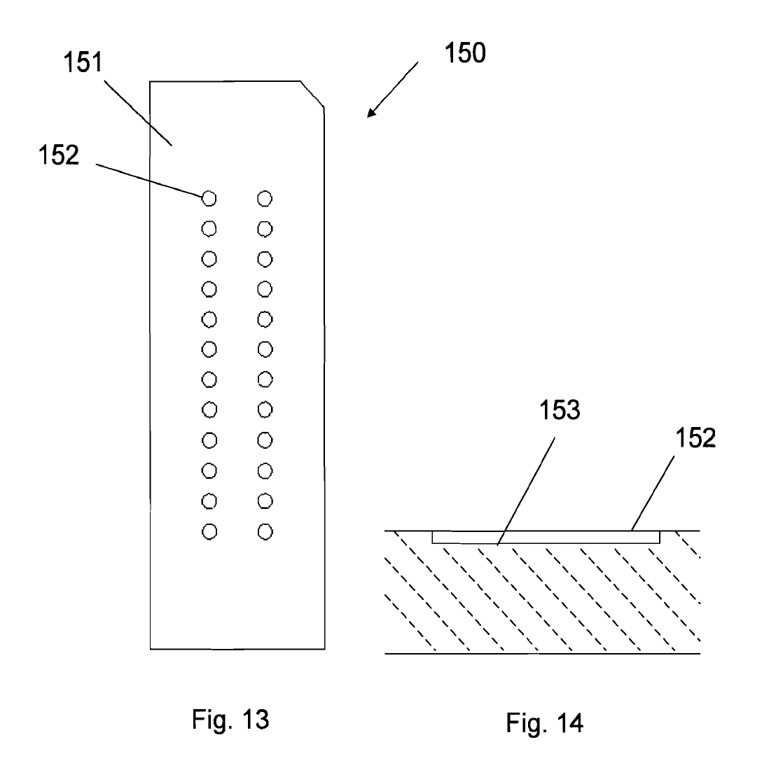

FIG. 13 shows an embodiment microtiter plate 150. FIG. 14 is a sectional view of one of the microtiter wells in the plate 150. The microtiter plate 150 provides a 12 by 2 array of wells 152. Of course, other dimensions of the array are possible, and thus other numbers of wells 152 can be created as determined, for example, from application requirements, such as 12 by 8, 24 by 16, or 48 by 32 arrays. The wells 152 in the embodiment 150 are 2 mm in diameter and 0.1 mm deep, but other dimensions can be used, such as 0.1 to 10 mm diameters and 0.1 to 10 mm depths, or as may be desired. The body 151 of the plate 150 is in a shape of 25×75 mm microscope slide with a thickness of 1 mm, which can be made from PDMS, polycarbonate, or other plastics. According to the size of the well array, other dimension of the plate 151 can be created. Functional materials, such as carbon nanotubes, can be embedded on the bottom portion of each well 153 using the methods described above. The microtiter plate can be optically transparent for UV/Vis absorbance detection or fluorescence detection, or black on the bottom for fluorescence detection from the top; therefore, it can be read by a fluorescence microscope, a microtiter plate scanner, a genearray scanner or the like.

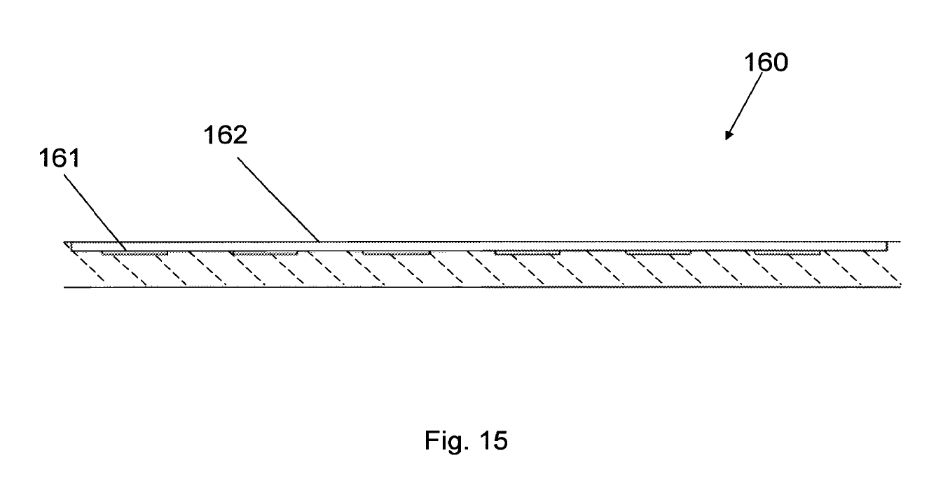

In other embodiments, a small pool can be created at the top of the wells, which can be used as a reservoir for sample reaction in the wells. FIG. 15 shows a sectional view of a device 160. The device 160 includes an array of wells 161 that are all set within a pool 162. The array of the wells can be divided into several groups by way of several pools 162 so that multiple samples can be analyzed on the device 160. A cover slip can be placed on top of the device 160 to form a chamber for sample reaction. Hence, groups of wells within respective pools can be fluidly isolated from each other on a single microtiter device.

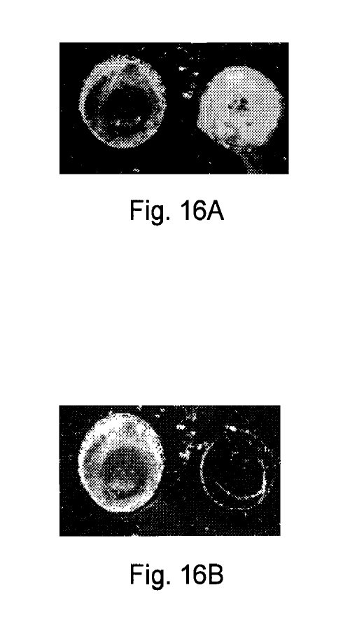

FIG. 16 shows two images from a fluorescence scanner, which demonstrates results obtained from a microtiter nanotube plate adapted for arsenite ion detection. For heavy metal ion detection, sensing molecules with optimal specificity are critical to success. Many researches are reporting on DNA, protein, and peptides used for metal ion detection purposes. For example, Ono et al. recently established a highly selective oligonucleotide-based sensor for Mercury ion (Hg2+) detection via the selective binding of Hg2+ to thymine-thymine (T-T) base pairs in DNA duplexes with a detection limit of 40 nM. Among the highly selective sensing molecules for heavy metal ions, peptides are particularly attractive because of their desirable stability and their combinatorial chemistry that can be used to find optimal amino acid sequences for specific heavy metal ion recognition. Peptides possess a variety of donor atoms for complexation to metal ions through an amino group, amide oxygen, amide nitrogen, and carboxyl oxygen. Some reports were published that use peptides for metal ion detection purposes. For example, NH2-Gly-Gly-His-COOH has been utilized for Copper ion (Cu2+) detection and has an extraordinarily low detection limit (sub-ppt) with minimal interference from other metal ions was reported; NH2-His-Ser-Gln-Lys-Val-Phe-COOH for Cadmium ion (Cd2+) has a detection limit of 5 nM; NH2-Cys-Pro-Gly-Cys-Lys-Lys-COOH for Arsenite ion (As3+) has a detection limit of 10 nM, and NH2-angiotensin-COOH for Lead ion (Pb2+) has a detection limit of 1.9 nM. In a specific embodiment, peptide nucleic acid (PNA: NH2-Glu-TTTTTTTTTTTTTTTTTTTTT-COOH) may be used for Mercury ion (Hg2+) detection.

To obtain the test results shown in FIG. 16, the steps described in the following may be performed:

1. Activation of nanotubes. Place a prepared activation solution containing 2 mM 1-ethyl-3-(3-dimethylaminopropyl)carbodiimide (EDC) and 5 mM N-hydroxysuccinimide (NHS) in 0.1 M 2-(N-morpholino)ethanesulfonic acid (MES) solution in wells of an embodiment device 150, or 160 for 30 minutes at room temperature.

2. Functionalization of nanotubes with sensing molecules. Dissolve the selected peptide NH2-Cys-Pro-Gly-Cys-Lys-Lys-COOH (1 mg/mL) in PBS buffer (pH 8-9). Place the solution in the wells for 60 minutes with shaker.

From the above steps, the peptide is coupled to the COOH group on the nanotubes at the bottom of the wells.

3. Fluorescence tag coupling. Apply the activation solution to the wells to activate the COOH groups on the peptide for 30 minutes, and then place the amino-terminated-fluorescence PBS solution in the wells for 60 minutes to link the fluorescence tag to the peptide.

4. Read the fluorescence intensity at each well. Scan the device and read the fluorescence intensities of the wells.

5. Sensing Arsenite ions. A prepared arsenic solution (100 ppb) is applied to the wells of the microtiter device for five minutes. The fluorescence intensity in each well of the device is read again.

FIG. 16 shows the changes in the fluorescence intensities at two wells. Magnetic beads were imbedded at the bottom of the well on the left for internal control, and carbon nanotubes on the right. FIG. 16A shows that the fluorescence intensities in the two wells after the coupling of the fluorescence tag, which are 458 at the magnetic bead well and 1100 at the nanotube well, with a ratio of 1:2.4. FIG. 16B shows the fluorescence intensities after the arsenic solution is applied to the wells for five minutes. The magnetic bead well gives a reading of 464 and the nanotube well a reading of 145, with a ratio of 1:0.31. The fluorescence signal is significantly quenched by the nanotubes due to the conformation change of the peptide after sensing the arsenite ions, which brings the fluorescence tag closer to the nanotubes.

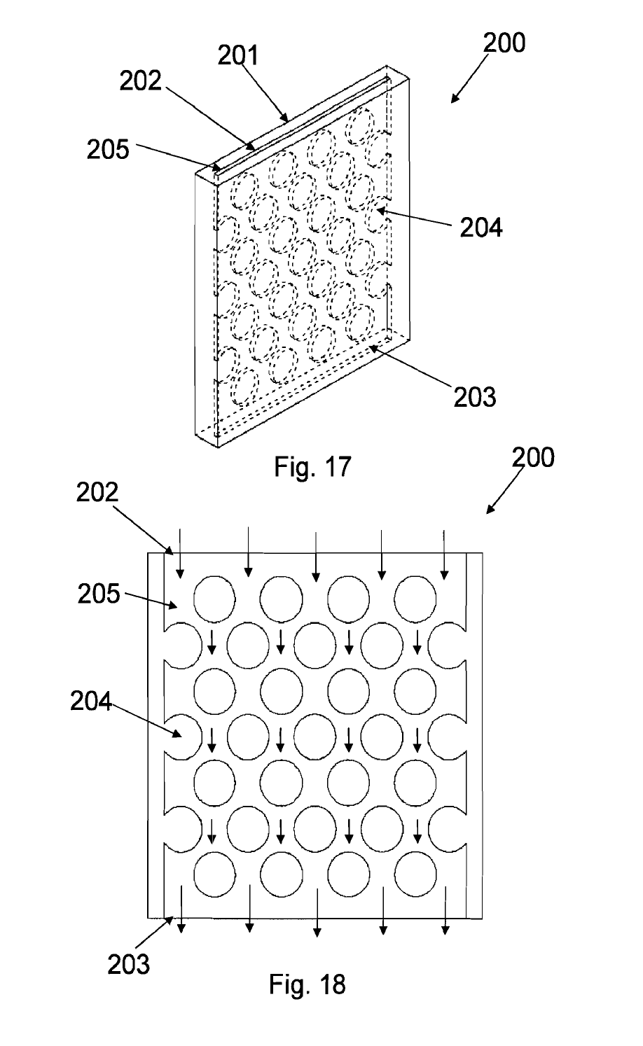

FIGS. 17 and 18 illustrate an embodiment suitable for use in a chemical or biochemical reaction vessel and filter vessel. The device 200 is a core component for such applications. The device 200 includes a body 201 that has a slot channel 205 with an inlet 202, an outlet 203 and an array of columns 204 disposed between the inlet 202 and outlet 203. A liquid sample can be injected via the inlet 202 into the slot channel 205, which then flows to the outlet 203. The column array 204 creates a turbulent flow of the liquid to ensure complete mixing of the sample inside the channel 205. Functional materials, such as nanotubes, may be embedded on the walls of the channel 205, surfaces of the columns 204 or both. For a reaction vessel, a catalyst can be bonded to the functional materials to accelerate a chemical or biochemical reaction as the liquid solution flows through the fluidic channel 205. For a filter vessel, sensing molecules can be bonded to the functional materials to capture targeted molecules that are to be removed from the liquid solution. The device 200 can be stacked and packed in an enclosure with an inlet and an outlet to increase the total liquid flow of the process.

Another aspect provides molecular imprinting to create a polymeric surface with template-shaped cavities (recognition elements) in polymer matrices providing a memory of the corresponding template molecules, which may be used in molecular recognition. Molecular imprinting is a process where functional and cross-linking monomers are co-polymerized in the presence of a target analyte (the imprinted molecule), which acts as a molecular template. In certain embodiments, functional monomers initially form a complex with the imprinted molecule, and following polymerization, their functional groups are held in position by the highly cross-linked polymeric structure. Subsequent removal of the imprinted molecule reveals binding sites that are complementary in size and shape to the analyte. In this way, a molecular memory is introduced into the polymer solution that provides an embodiment functional solution and which is capable of rebinding the analyte with a very high specificity. The imprinted solution may be disposed on any desired surface of the sacrificial mold, and may be disposed on more than one surface, or in more than one region and then, by the molding process described above, transferred to a polymeric surface to provide a functional surface or a functional spot.

As an example embodiment of molecular imprinting, detection of melamine in an aquatics solution is presented as follows:

1. Polymerize monomers with template molecules:
1) Heat 20 ml of DI water to 60° C. and then add 0.1 g of melamine powder (i.e., the template molecule) into the heated DI water.
2) Stir the mixture until the melamine powder totally dissolves.
3) Add 0.2 g of 3-aminobenzoic acid and 0.05 g of aniline (monomers) into the above solution, and stir until the powder totally dissolves.
4) Add 1 ml of an oxidizing agent solution (such as 0.1 g of ammonium persulfate in DI water) to initialize the reaction.
5) The reaction is carried out at room temperature for 7 hours with constant stirring.

2. Remove the template molecule from the above solution to form an imprinting functional polymer solution:
1) From the above step 1.5, stop stirring and let the polymer precipitate.
2) Take the upper solution out, leaving the polymer precipitate.
3) Add a similar amount of DI water to the polymer precipitate and stir the solution for 20 minutes.
4) Repeat the above steps 2.1-2.3 three times.

3. Replace the water with a solvent:
1) From the above step 2.4, remove the upper solution to leave the polymer precipitate.
2) Add a similar amount of solvent, such as ethanol, to the polymer precipitate and stir the solution for 10 minutes.
3) Repeat the above steps 3.1-3.3 two times.

4. Make a functionalized device, such as a cuvette, using the molecular imprinting solution from the above steps 1-3 as the functional solution.

The procedure to make a functionalized device, such as a cuvette, may follow the description provided earlier for the making of devices with functionalized surfaces, in which the above molecular imprinting solution is used as the functional solution. The resultant device, such as a cuvette or a microtiter plate, is functionalized for detection of the template molecule, such as in the above example for melamine. It will be appreciated that the instant cuvette is a preferred example for molecular imprinting applications, but other formats, such as microtiter plates, are also applicable to embodiments of this application.

Testing a sample having the target material:

The protocol to test a sample, for example such as a sample with melamine, can follow the description provided above with respect to the testing for Fe3+. An embodiment cuvette with a functional surface imprinted for detection of melamine as described above was created and had a sensitivity of 1 ppm with a UV absorption peak at 263 nm. The melamine absorption peak is at A=236 nm, whereas the absorption of the melamine molecular imprinted cuvette was found to be at 263 nm. Without wishing to be bound by theory, it is believed that the red shift is probably due to interactions between melamine molecules and the polymer matrix.

Advantages of the various embodiment devices and related methods include:

1. Low cost. The nano-engineered or functional surfaces are made from a sacrificial mold material process, which is far less expensive than the current nanofabrication or surface modification technologies.

2. Small amounts and flexible amounts of a sample are needed. The samples used with embodiment devices can be from, for example, nanoliters to milliliters in volume.

3. Easy and simple handling. Enzymes or molecules can be attached to the functional surface, such as a functional surface provided by carbon nanotubes, just prior to use or in the manufacturing process. Measurement can be performed in just one step, which simply involves adding the sample solution into the upper compartment of the cuvette or the wells of a microtiter device.

4. High sensitivities. Highly dense binding sites for targeted molecules on the functional surface, such as functionalized nanomaterials (i.e., carbon nanotubes), and flow-through sample solution significantly increase the detection sensitivities.

5. High speed. The molecular binding reaction is very rapid between the sensing molecules on the functional surfaces and the targeting molecules in the sample. Because of the nanomaterials and the functional groups on their surfaces that increase the sensing area and the density of the sensing molecules, the time for target measurement can be less than 1 minute.

6. Embodiments may be utilized as a facile reaction vessel with functional surfaces embedded on the walls of flow channels for separation of final products or for catalysts. Embodiments may also be used as a “smart filter” for molecules with small or similar sizes of the product molecules, where specific enzymes are immobilized on functional surfaces, such as on nanotubes, that are embedded on the wall of the filter flow pathway.

The invention described herein of embedding functional materials on a surface can be used for any targeted molecules that exhibit optical signal changes during a sensing processes and can physically or chemically, such as through an intermediate molecule, attach onto functional materials, such as carbon nanotubes; examples include fluorescent sensors (4-aminophthalimide derivatives or peptides) for transition metal ions, polymeric optical sensors for organic aromatic compounds, and indicator dyes for the detections of amines, humidity, alcohols, fructose, glucose, formaldehyde, etc. Embodiments may be useful in analytical chemistry, biological diagnosis, medical diagnosis, food testing, environment testing, bio-defense, drug detection, and combination chemistry for drug development. Although some examples have been discussed above, other implementation and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A method for making a functionalized surface, comprising:
contacting a functional solution with a sacrificial material;
disposing a liquid polymer onto the sacrificial material;
allowing the liquid polymer to cure; and
dissolving the sacrificial material from the cured polymer.

2. The method of claim 1 further comprising dispersing or dissolving functional materials into a solvent to create the functional solution.

3. The method of claim 2 wherein the functional material comprises chemically processed nanomaterials, palmitic acid, polyhydroxystyrene, polyacrylic acid, polycarbonate resin, or combinations thereof.

4. (canceled)

5. The method of claim 1 wherein the functional solution is a molecular imprinting solution comprising template molecules polymerized with monomers.

6. The method of claim 1 wherein the sacrificial material is made from a water-soluble or a solvent-soluble material.

7. The material of claim 6 wherein the water-soluble or solvent-soluble material is selected from one or more of the set consisting of polyvinyl alcohol (PVA), starch, gelatin, synthetic polymers, colloid gels, and lipid materials.

8. The method of claim 1 wherein the sacrificial material comprises a porous surface, wherein a pore size of the porous surface is from 10 to 1000 nanometers in size.

9. The method of the claim 1 wherein depositing the functional solution onto the surface of the sacrificial material is performed by pipetting the functional solution onto the surface of the sacrificial material or by dipping the sacrificial material into the functional solution.

10. The method of the claim 1 further comprising depositing a plurality of different types of functional solutions onto surfaces of the sacrificial material to create multiple functional surfaces with different respective functionalized properties.

11. (canceled)

12. (canceled)

13. A fluidic device comprising:
an enclosure body;
an inlet in the enclosure body;
an outlet in the enclosure body;
a fluidic channel in the enclosure body fluidly connecting the inlet to the outlet; and
a functional surface in the fluidic channel.

14. The fluidic device of claim 13 further comprising an orifice to control a flow rate of fluid within the fluidic channel.

15. The device of the claim 13 wherein the fluidic channel comprises a plurality of columns.

16. (canceled)

17. The fluidic device of claim 13 further comprising a filling disposed within the enclosure body, the filling defining the fluidic channel.

18. The fluidic device of claim 17 wherein the filling is made from one or more of polydimethylsiloxane (PDMS), polyurethane, polydimethylsiloxane, polycarbonate, polypyrrole, resin, Teflon resin, epoxy, polymeric rubber, and polymeric plastic.

19. The device of the claim 13 wherein the functional surface is disposed on a sidewall of the fluidic channel which is substantially non-parallel to a detection light pathway or to an excitation light pathway.

20. The device of the claim 13 wherein the functional surface comprises nanomaterials, palmitic acid, polyhydroxystyrene, polyacrylic acid, polycarbonate resin, or combinations thereof, or a molecular imprint of a target analyte.

21. The device of the claim 13 further comprising a plurality of functional surfaces disposed on respective sidewalls of the fluidic channel with different functional materials.

22. (canceled)

23. A microtiter device comprising:
a body; and
a plurality of spots disposed in the body, each of the plurality of spots comprising a functional surface.

24. The microtiter device of claim 23 wherein the functional surfaces comprise one or more of nanomaterials, palmitic acid, polyhydroxystyrene, polyacrylic acid, polycarbonate resin, or imprinted molecules of a target analyte.

25. The microtiter device of claim 23 wherein the spots are wells, the functional surfaces are disposed on bottom surfaces of the wells, and the wells having a depth of from 0.1 mm to 10 mm, and diameters from 0.1 mm to 10 mm.

26. (canceled)

27. (canceled)
